(19) United States
(12) Patent Application Publication
(10) Pub. No.: US 2022/0241090 A1
(43) Pub. Date: Aug. 4, 2022

(54) MUSCLE LENGTH MEASUREMENT

(71) Applicant: Mayo Foundation for Medical Education and Research, Rochester, MN (US)

(72) Inventors: Alexander W. Hooke, Rochester, MN (US); Joaquin Sanchez-Sotelo, Rochester, MN (US); Bassem T. Elhassan, Rochester, MN (US)

(21) Appl. No.: 17/623,669

(22) PCT Filed: Jul. 8, 2020
(86) PCT No.: PCT/US20/41136

§ 371 Date: Dec. 29, 2021

Related U.S. Application Data
(60) Provisional application No. 62/880,813, filed on Jul. 31, 2019.

(51) Int. Cl. A61F 2/46; A61F 2/40; G16H 20/40

(52) U.S. Cl. CPC A61F 2/46; A61F 2/40; A61F 2002/30948; A61F 2002/4633; A61F 2002/4658; G16H 20/40

(57) ABSTRACT

Operative lengths of muscles can be determined. For example, this document describes techniques for measuring operating muscle lengths of the infraspinatus, teres minor, and subscapularis through the internal-external rotation range of motion before and after implantation of RSA components. In some embodiments, the techniques described herein can be advantageously used as a part of preoperative surgical planning Such pre-surgical planning can help to select optimal prosthetic implants, ensure fewer surgical complications, and attain better patient outcomes.

### CROSS-REFERENCE TO RELATED APPLICATIONS

This application claims the benefit of U.S. Provisional Application Ser. No. 62/880,813, filed Jul. 31, 2019. The disclosure of the prior application is considered part of (and is incorporated by reference in) the disclosure of this application.

### BACKGROUND

### 1. Technical Field

This document relates to techniques for measuring the operative lengths of muscles. For example, this document relates to techniques for measuring operating muscle lengths of the infraspinatus, teres minor, and subscapularis through the internal-external rotation range of motion before and after implantation of reverse shoulder arthroplasty components.

### 2. Background Information

Surgical planning is the preoperative method of simulating and pre-visualizing a surgical intervention in order to predefine surgical steps and to select optimal types and sizes of prosthetic implants in the context of the final outcome. In some cases, three-dimensional (“3D”) images of surgical sites that are useful for surgical planning can be obtained using computed tomography (“CT”) imaging, for example.

Reverse shoulder arthroplasty (“RSA”) has been reported to result in changes in the length, tension and line of action of the rotator cuff muscles. The magnitude and direction of these changes are influenced by the geometry of the implant design such as, but not limited to, the degree of lateralization of the glenoid and humeral implant components, the diameter of the glenosphere, and the tilting of glenoid and humeral components. One proposed advantage of lateralized implants is the increase in length and tension of the rotator cuff, which could position these muscles in an optimal part of their force-length curve. It would be desirable to preoperatively measure or estimate operating length changes of axial rotator cuff muscles associated with implantation of varied RSA designs and sizes.

### SUMMARY

This document describes techniques for measuring the operative lengths of muscles. For example, this document describes techniques for measuring operating muscle lengths of the infraspinatus, teres minor, and subscapularis through the internal-external rotation range of motion before and after implantation of RSA components.

In one implementation, a pre-surgical planning method for a patient receiving a prosthetic implant is disclosed herein. The method includes: (i) identifying, in an image, origin and insertion locations of a muscle; (ii) modeling, using software and the image, rotation of a joint that is simulated based on a computer model of a potential prosthetic implant; (iii) recording coordinate data of the origin and insertion locations during the modeled rotation of the joint; and (iv) determining, using the recorded coordinate data, an actual prosthetic implant to be surgically implanted in the patient.

Such a method may optionally include one or more of the following features. In some embodiments, the muscle is an infraspinatus, teres minor, or subscapularis muscle. The joint may be a shoulder joint. The method may also include determining a maximum length of the muscle. The method may also include determining an arc length of the muscle that is wrapped around a bone. The determining the actual prosthetic implant to be surgically implanted in the patient may include selecting a particular type or size of implant for which a calculated operative length of the muscle is the closest to a desired operative length of the muscle. In some embodiments, the image is a three-dimensional computed tomography image.

Particular embodiments of the subject matter described in this document can be implemented to realize one or more of the following advantages. In some embodiments, the techniques described herein can be advantageously used as a part of preoperative surgical planning. Such pre-surgical planning can help to ensure fewer surgical complications and better patient outcomes. In some embodiments, the techniques described herein facilitate simulations of the effects of various types of prosthetic implants on the patient's post-operative musculoskeletal system. For example, in the context of RSA, the techniques described herein can be used to compute the muscles' operative length for each muscle acting as an axial rotator of the shoulder. Moreover, the modeling and simulation can be carried out for multiple types and/or sizes of prosthetic ball-and-socket joint implants. By comparing and contrasting such simulation models, a surgeon can select an optimal type/size of prosthetic ball-and-socket joint implant for a particular patient.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram depicting a musculoskeletal system of a portion of a human shoulder joint in a first arrangement.

FIG. 2 is a schematic diagram of the musculoskeletal system of the portion of a human shoulder joint of FIG. 1 in a second arrangement.

FIG. 3 is flowchart of a method for measuring the operative lengths of muscles in accordance with some embodiments provided herein.

FIG. 4 is a graph showing the results of experiments involving measuring ranges of muscle length during axial shoulder rotation.

Like reference numbers represent corresponding parts throughout.

### DETAILED DESCRIPTION

This document describes techniques for measuring the operative lengths of muscles. For example, this document describes techniques for measuring operating muscle lengths of the infraspinatus, teres minor, and subscapularis through the internal-external rotation range of motion before and after implantation of RSA components. In some embodiments, the techniques described herein can be advantageously used as a part of preoperative surgical planning. Such pre-surgical planning can help to select optimal prosthetic implants, ensure fewer surgical complications, and attain better patient outcomes.

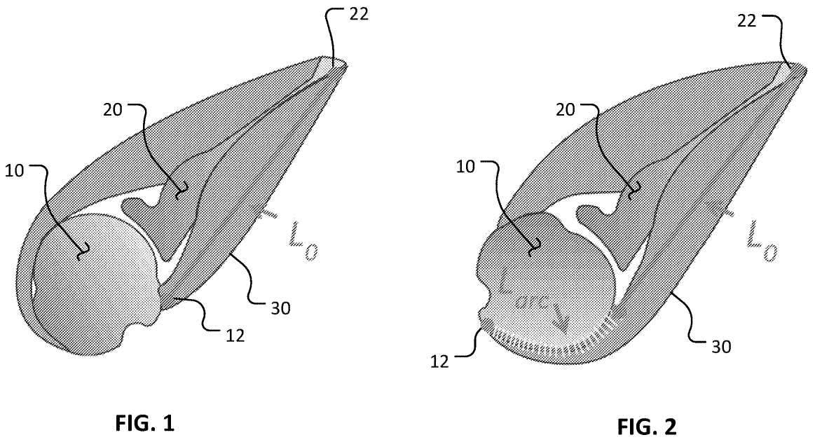

FIGS. 1 and 2 depict the musculoskeletal system of a portion of a human shoulder joint, including a humerus bone 10, a scapula 20, and a subscapularis muscle 30. The subscapularis 30 is the largest, strongest muscle of the rotator cuff. The rotator cuff muscles are important in shoulder movement and help maintain glenohumeral joint stability.

While the subscapularis 30 is used in this disclosure as an example, it should be understood that the principles described herein are applicable to other shoulder muscles (e.g., infraspinatus, teres minor), and to other muscles of the human anatomy in general.

The subscapularis 30 lies at the anterior surface of the scapula 20. The subscapularis 30 originates at the subscapular fossa, and specifically the medial and lower two-thirds of the groove on the lateral border of the scapula 20. The subscapularis 30 inserts at the lesser tubercle of the humerus 10.

In the arrangement of FIG. 1, the humerus 10 is in its maximum interal rotation position, which puts the subscapularis 30 is in its minimum muscle length position (labeled “L₀”). In the arrangement of FIG. 2, the humerous 10 has been moved to its maximum external rotation position, so that the subscapularis 30 is in a maximum muscle length position (“Lₘₐₓ”). The length of the subscapularis 30 in its minimum muscle length portion (FIG. 1) and the length of the subscapularis 30 in its maximum muscle length portion (FIG. 2) define the operative of the subscapularis 30.

For each muscle acting as an axial rotator of the shoulder, the following steps can be taken to compute the muscles' operative length. Muscle operative length is defined as the range of lengths a muscle will operate in over its internal/external axial range of motion while maintaining a constant abduction and flexion/extension position.

The three-dimensional xyz coordinates of the location of the origin of the subscapularis 30 must be identified in a coordinate system fixed to the bone on which the muscle originates (i.e., the scapula 20). The origin location is labeled in FIGS. 1 and 2 as origin 22.

Additionally, the three-dimensional xyz coordinates of the location of the insertion of the subscapularis 30 must be identified in a coordinates system fixed to the bone on which the muscle inserts (i.e., the humerus 10). The insertion location is labeled in FIGS. 1 and 2 as insertion 12.

The shoulder joint is then moved through its full internal/external axial rotation range of motion while motion about the abduction and flexion/extension axes is held constant. During this motion, the three-dimensional position and orientation of the origin 22 and the insertion 12 must be tracked in a global coordinate system such that bone-to-global transformation matrices are created. These transformation matrices are used to transform the muscle's origin 22 and insertion 12 local xyz coordinates into xyz coordinates in the shared, global coordinate system.

After the global xyz coordinates for the origin 22 and insertion 12 are computed for each position of the shoulder during its internal/external axial rotation range of motion, the operative length can be computed.

When the shoulder is at its end range of motion with the subscapularis muscle 30 at its shortest length (internal rotation, as depicted in FIG. 1), the linear distance between the origin 22 and insertion 12 is computed, referred to as minimum length L₀.

As the shoulder moves from this minimum length position through its axial rotation range of motion to its maximum length position (external rotation, as depicted in FIG. 2), the length of the arc traced by the insertion point 12 as it wraps around the proximal humerus 10 is computed as excursion (or “L_{arc}”).

The maximum length Lₘₐₓ of the subscapularis muscle 30 is then computed as the sum of these two components: L₀ and L_{arc}. In other words, Lₘₐₓ=L₀+L_{arc}. The subscapularis muscle 30 operative length is defined as the range from L₀ to Lₘₐₓ.

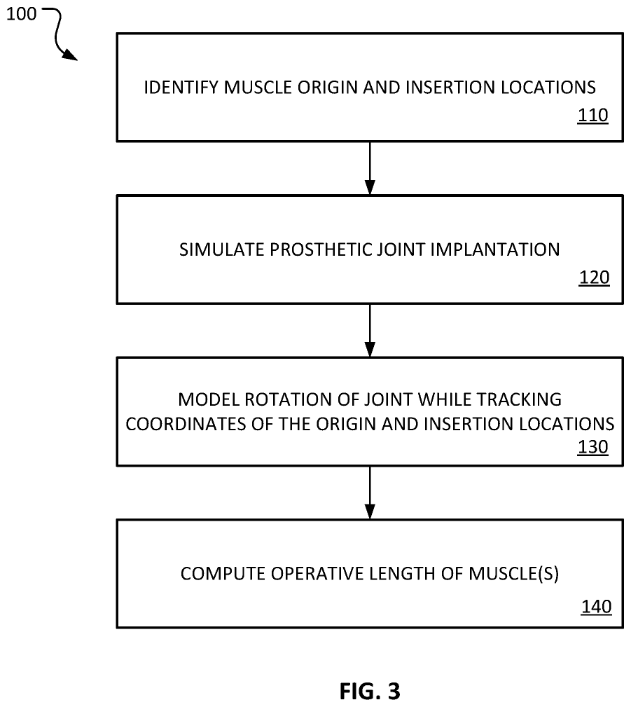

The techniques for determining a muscle's operative length can be used, for example, in an example method 100 for computing operative muscle lengths as described in the flowchart of FIG. 3.

In step 110, the origin and insertion locations of a particular muscle are identified as landmarks that will be tracked during the method 100. For example, in some cases one or more CT scans of the bones (e.g., the humerus 10 and the scapula 20, or other bones) can be obtained and loaded into software (e.g., a surgical planning software). Using the software, the origin and insertion landmarks of the muscle(s) can be identified, and the coordinates of the origin and insertion locations can be calculated in the software.

In step 120, a computer model of a prosthetic implant can be loaded into the software (e.g., surgical planning/modeling software) and applied in conjunction with the CT scan(s) from step 110.

In step 130, the software is used to simulate movement of the joint through a range of motion (using the computer model of the implant). For example, the humerus 10 can be simulated as moving around in the joint by rotation of the humerus 10 about its internal and external axes of rotation (e.g., through its arc of motion). As described above, the muscle (e.g., subscapularis 30) wraps around the bone (e.g., the humerus 10) during the rotation. During the simulated rotation, the coordinates of the origin and insertion landmarks are recorded or tracked and collected.

In step 140, the data of the positions of the origin and insertion landmarks from step 130 are used. In some cases, the landmark data is exported to other software for analysis. Using the data, for example, the muscle's operative length and other relevant parameters can be determined (as described above). For instance, the excursion as the insertion point 12 as it wraps around the proximal humerus 10 is can be computed as excursion (or “L_{arc}”), as can other useful parameters.

In some cases, the method 100 (or portions thereof) may be repeated for another type or size of implant. The results can be compared and evaluated. A surgeon can use the results for surgical preplanning and/or for selection of a suitable type or size of implant. In some cases the method 100 can be used to select a particular type or size of implant for which the muscle is the closest to the natural operative muscle length.

### EXPERIMENTAL STUDIES

A cadaver model was utilized to determine operating muscle lengths of the infraspinatus, teres minor, and subscapularis through the internal-external rotation range of motion before and after implantation of reverse shoulder arthroplasty (RSA) components with a lateralized glenosphere. All three muscles show increased minimal and maximal lengths and an increased total excursion following RSA.

RSA has been reported to result in changes in the length, tension and line of action of the rotator cuff muscles. The magnitude and direction of these changes is influenced by a combination of the implant's components design factors. One proposed advantage of lateralized implants is the increase in length and tension of the rotator cuff, which could position these muscles in a desired part of their force-length curve. The purpose of this study was to measure operating length changes of axial rotator cuff muscles with implantation of an RSA that lateralizes on both the glenoid and humeral sides.

A cadaveric model of six hemi-thoraces was used. The spine, pelvis, sternum, rib cage, humerus and all muscles of the thorax, back, and shoulder girdle were preserved. Suture lines were run to pneumatic cylinders from the insertion to the origin (guided by eyelets screwed to bones) of ten muscles to apply a constant, stabilizing load across the joint.

Electromagnetic tracking sensors were rigidly fixed to the thorax, scapula, and humerus to record 3D kinematics with anatomic coordinate systems established according to ISB recommendations. Muscle origins and insertions were digitized and tracked.

Testing consisted of manually rotating the humerus through five cycles of its internal-external rotation range of motion. Kinematic data, including the origin and insertion coordinates, was collected at 120 Hz using the Motion Monitor software. Testing was performed in three positions of abduction: 0°, 45°, and 90°. After testing the intact shoulder, reverse shoulder arthroplasty was performed by a trained orthopedic surgeon using an implant with 2 mm of glenosphere lateral offset and testing was repeated.

Muscle lengths of the internal (subscapularis) and external (infraspinatus and teres minor) rotators were computed by taking the linear origin to insertion distance at each muscle's shortest point plus the increasing arc length of the insertion point as it wrapped the proximal humerus during axial rotation. A matched pairs t-test (p=0.05) was performed comparing the intact and RSA conditions for each muscle.

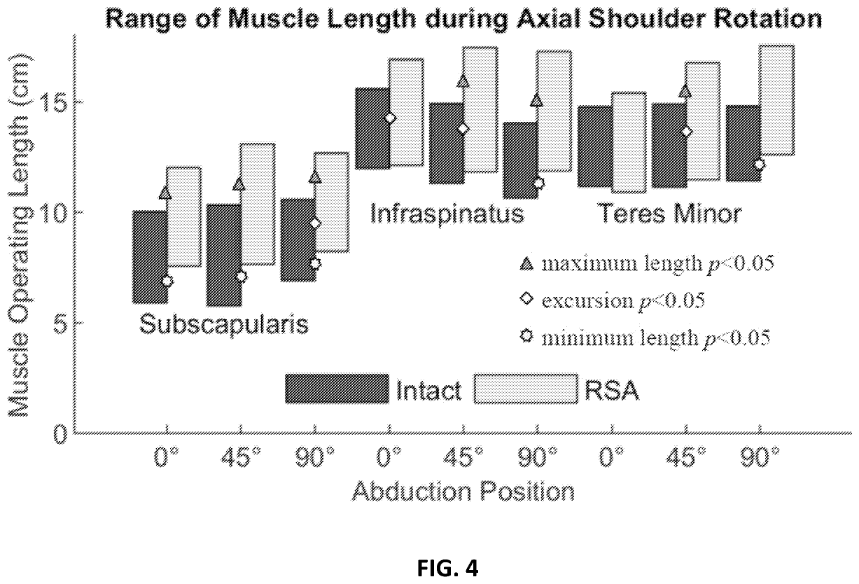

Referring to the results shown in FIG. 4, subscapularis minimum and maximum lengths increased after RSA across all abductions and increased excursion at 90° abduction. Infraspinatus increased in minimum length at 45° and 90°, in maximum length at 90, and in excursion at 0° and 45°, after RSA. Teres minor minimum and maximum lengths increased after RSA at 90° and 45°, respectively, and excursion was increased at 45° after RSA. The bottoms and tops of the bars in the graph indicate minimal and maximal muscle lengths, respectively. Bar size indicates total excursion.

Increased minimal length was likely due to the combined glenoid and humeral lateralization. While the implant used was lateralized compared to the Grammont-style implants, the center of rotation remained medial relative to the native shoulder. This medialization of the center of rotation relative to the lateral humeral surface increases the radius of humeral axial rotation axis versus the intact arm, increasing the maximum length and excursion of the cuff muscles. Given the known force-length relationship of muscles, this change in the operating lengths will likely affect shoulder function.

This experiment shows that RSA with a lateralized implant increases the operating lengths of the subscapularis, infraspinatus, and teres minor, thereby altering their biomechanical function and force generating capabilities.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A pre-surgical planning method for a patient receiving a prosthetic implant, the method comprising:
identifying, in an image, origin and insertion locations of a muscle;
modeling, using software and the image, rotation of a joint that is simulated based on a computer model of a potential prosthetic implant;
recording coordinate data of the origin and insertion locations during the modeled rotation of the joint; and
determining, using the recorded coordinate data, an actual prosthetic implant to be surgically implanted in the patient.

2. The method of claim 1, wherein the muscle is an infraspinatus, teres minor, or subscapularis muscle.

3. The method of claim 1, wherein the joint is a shoulder joint.

4. The method of claim 1, further comprising determining a maximum length of the muscle.

5. The method of claim 4, further comprising determining an arc length of the muscle that is wrapped around a bone.

6. The method of claim 1, wherein the determining the actual prosthetic implant to be surgically implanted in the patient comprises selecting a particular type or size of implant for which a calculated operative length of the muscle is the closest to a desired operative length of the muscle.

7. The method of claim 1, wherein the image is a three-dimensional computed tomography image.

8. A pre-surgical planning system configured to:
identify, in an image, origin and insertion locations of a muscle;
model, using software and the image, rotation of a joint that is simulated based on a computer model of a potential prosthetic implant;
record coordinate data of the origin and insertion locations during the modeled rotation of the joint; and
determine, using the recorded coordinate data, an actual prosthetic implant to be surgically implanted in the patient.

9. A pre-surgical planning system configured to facilitate:
identifying, in an image, origin and insertion locations of a muscle;
modeling, using software and the image, rotation of a joint that is simulated based on a computer model of a potential prosthetic implant;
recording coordinate data of the origin and insertion locations during the modeled rotation of the joint; and
determining, using the recorded coordinate data, an actual prosthetic implant to be surgically implanted in the patient.
